(19) United States
(12) Patent Application Publication
(10) Pub. No.: US 2011/0297904 A1
(43) Pub. Date: Dec. 8, 2011

(54) HUMIDITY RESPONSIVE MATERIALS AND SYSTEMS AND METHODS USING HUMIDITY RESPONSIVE MATERIALS

(75) Inventors: Ali Dhinojwala, Akron, OH (US); Todd Blackledge, Akron, OH (US); Ingi Agnarsson, Stow, OH (US)

(73) Assignee: THE UNIVERSITY OF AKRON, Akron, OH (US)

(21) Appl. No.: 12/990,406

(22) PCT Filed: May 1, 2009
(86) PCT No.: PCT/US09/42583

§ 371 Date: Oct. 29, 2010

Related U.S. Application Data
(60) Provisional application No. 61/049,991, filed on May 2, 2008.

(51) Int. Cl. B66B 1/00; C07K 14/00; C07K 14/435

(52) U.S. Cl. 254/387; 530/353; 442/1

(57) ABSTRACT

The invention relates to silk or other materials formed to have predetermined contraction/relaxation characteristics, wherein the contraction/relaxation characteristics are initiated by exposure thereof to predetermined humidity characteristics in the adjacent atmosphere. The materials may comprise a single silk fiber, a bundle of fibers of a predetermined size or diameter, a meshwork of fibers forming a predetermined configuration such as one or more sheets, bundles or other bodies. In this manner, the material can be scaled across a size range of any desired magnitude to produce predetermined force and/or displacement characteristics in association therewith.

### CROSS REFERENCE TO RELATED APPLICATION

The present application is being filed with the U. S. Receiving Office as a PCT application claiming priority from and any other benefit of U.S. provisional patent application Ser. No. 61/049,991 filed on May 2, 2008, the entire disclosure of which is hereby incorporated by reference.

### FIELD OF INVENTION

The present invention relates to humidity responsive materials and systems and methods using materials which exhibit cyclical responses to changes in humidity. More specifically, the invention is directed to silk materials having predetermined cyclical responses of contraction and relaxation, and systems and methods using the silk material having predetermined contraction/relaxation characteristics in response to changes in humidity.

### BACKGROUND OF THE INVENTION

The graceful motion of a cheetah sprinting after a gazelle or a basilisk lizard running across the water provide elegant examples of how biological muscles provide large forces in a short time. To date, synthetic mimics of biological muscles generally lack the same dexterity and energy density exhibited by biological muscle. Moreover, most biomimetic muscles cannot scale effectively across large ranges in cross-sectional areas, limiting their applicability. Further, synthetic muscles developed so far are driven by electric voltage, and thus require electrodes and large external power supplies. For example, polymer-based synthetic muscle mimics are activated by electric fields (E=V/d). In order to keep the driving voltages (V) low, the devices have to be 100's μm thin (d) and many layers have to be stacked together to scale up the devices to larger dimensions. Therefore, there is a need in the art for a system which may, as an example, be used to mimic biological muscle which does not require activation by electric fields and is not created by advanced processing.

For a variety of other applications and systems, there is a need to apply a force on one or more elements of the system, and/or to cause displacement of one or more elements. In many systems and applications, force or displacement is provided by electrical motors or systems requiring external power supplies. It would be desirable to allow force or displacement to be achieved in a simple and effective manner across a large scale of sizes and magnitudes without requiring electrical energy.

Most of the world's 40,000 species of spiders, including the golden silk orbweaver Nephila clavipes, produce dragline silk from major ampullate silk glands to spin lifelines and frames of webs. In addition to dragline silk, spiders may also produce other silks for different functions, each having different physical and mechanical properties, including sticky capture silk used for capturing prey within a web, silk for wrapping captured prey, silk for producing egg sacs, and piriform silk for connecting other types of silk together. In comparison to the other types of silk, dragline silk has the highest tensile strength and has among the largest diameters.

Dragline silk's impressive toughness (work/volume approximately five times greater than Kevlar®), high strength to weight ratio (approximately five times greater than steel), immunological compatibility with living tissue, and production under environmentally benign conditions all make spider silk a unique material.

It has been noted that silk material contracts with changes in humidity Furthermore, because this effect already operates on the scale of single silk fibers, which may be sub-micron to as big as five μm in diameter, it can easily be scaled across a wide size range.

### SUMMARY OF THE INVENTION

The invention relates to silk or other materials formed to have predetermined contraction/relaxation characteristics, wherein the contraction/relaxation characteristics are initiated by exposure thereof to predetermined humidity characteristics in the adjacent atmosphere. The materials may comprise a single silk fiber, a bundle of fibers of a predetermined size or diameter, a meshwork of fibers forming a predetermined configuration such as one or more sheets, bundles or other bodies. In this manner, the material can be scaled across a size range of any desired magnitude to produce predetermined force and/or displacement characteristics in association therewith.

In another aspect of the invention, the material is formed to produce a biomimetic fiber product, using one or more fibers forming a muscle-emulating body scaled to a predetermined size to have predetermined contraction/relaxation characteristics to impart predetermined forces between biological members, so as to operate in a manner emulating biological muscle operation. In a further aspect of the invention, there is provided a system having at least two members between which at least one biomimetic fiber is attached to impart a predetermined force or displacement between the at least two members, as provided by predetermined contraction/relaxation characteristics of the at least one biomimetic fiber. The system further comprises a controlled atmosphere at least in the vicinity adjacent the at least one fiber, such that the predetermined contraction/relaxation characteristics of the at least one biomimetic fiber are controllable by control of the humidity in the atmosphere adjacent the at least one biomimetic fiber.

The invention is also directed to methods of using at least one fiber by attaching the at least one fiber to at least two body members to selectively impart a force on at least one body member or displacement of at least one body member by subjecting the at least one fiber to an atmosphere having predetermined humidity characteristics, wherein the predetermined humidity characteristics cause predetermined contraction or relaxation of the at least one fiber. In the systems and methods of examples of the invention, the simplicity of using wet or dry air to drive the biomimetic silk fibers between predetermined contracted or relaxed positions can provide power generated by the contraction/extension of the material to provide unique possibilities in designing light weight and compact actuators, sensors, energy production systems and other systems or methods for a variety of applications.

### BRIEF DESCRIPTION OF THE DRAWINGS

FIG. 1 shows the response of a single 5 μm diameter dragline fiber from the golden silk spider Nephila clavipes to cyclical changes in humidity;

FIG. 2 depicts a graph showing the relationship between the magnitude of the stress generated as a function of the change in humidity; depicts a graph showing power generated as a function of applied stress (normalized by cross-sectional area of the spider silk);

FIG. 3 shows show cyclic lifting of a 9.5 mg weight in response to seven cycles of drying in an environmental chamber;

FIG. 4 shows a graph of the power density vs. stress;

FIG. 5 depicts a graph showing the relationship between the amount of lift force generated versus the number of bundled silk fibers;

FIG. 6 depicts a graph showing the cyclical behavior of contraction forces in relation to relative humidity for a variety of silk fiber bundles;

FIG. 7 illustrates an embodiment of a system comprising a humidity responsive material as presently disclosed;

FIG. 8 illustrates a further embodiment of a system comprising a humidity responsive material as presently disclosed; and

FIG. 9 is a graph showing maximum stress vs. modulus for spider silk, silkworm silk and other materials.

### DETAILED DESCRIPTION OF THE INVENTION

Examples of the materials according to aspects of the invention and of systems and methods according to the invention will be described below, with reference to various Figures. In general, the invention is directed to synthesized materials having the characteristics similar to silk materials produced by spiders or other silk producing organisms. For example, synthetic silk may be manufactured by combining proteins in a laboratory environment. As another example, synthetic silk may be produced by organisms other than spiders which carry spider silk-producing genes, for example silkworms or goats. However, it is also envisioned that natural silk produced by spiders may be utilized in any of the exemplary embodiments. In a desired form, the material may have mechanical properties similar to the dragline silk produced by various spider species, with high tensile strength characteristics, mechanical anisotropy in the fiber, and dynamic stiffness characteristics as an example. Other forms of silk produced by silk producing organisms may also be desirably incorporated into the material for use in various specific applications or environments. A synthesized material may therefore be an extracellular fibrous protein with characteristics of strength and elasticity emulating silk as produced by such organisms. The silk fiber may comprise pseudocrystalline regions of antiparallel B-sheet interspersed with elastic amorphous segments. The repetitive sequence of proteins, such as a fibroin protein, may form the fibrous material. The physical properties may be based on the properties of fiber formation, strength, and elasticity, based on the repetitive protein sequence. In some such silk materials, one of the properties is that the fiber may exhibit behavior known as supercontraction. At a threshold humidity, an unrestrained silk fiber may contract up to 50% (or more) in length while restrained silk may generate force or stresses in excess of 50 MPa based on the supercontraction characteristics of the fiber. Supercontraction, however, is typically a one-time response to a threshold humidity level or higher humidity in a restrained silk fiber. In contrast to supercontraction, it has also been found that silk fiber can also exhibit a distinct response to humidity levels, which permits the silk fiber to repeatedly contract and relax.

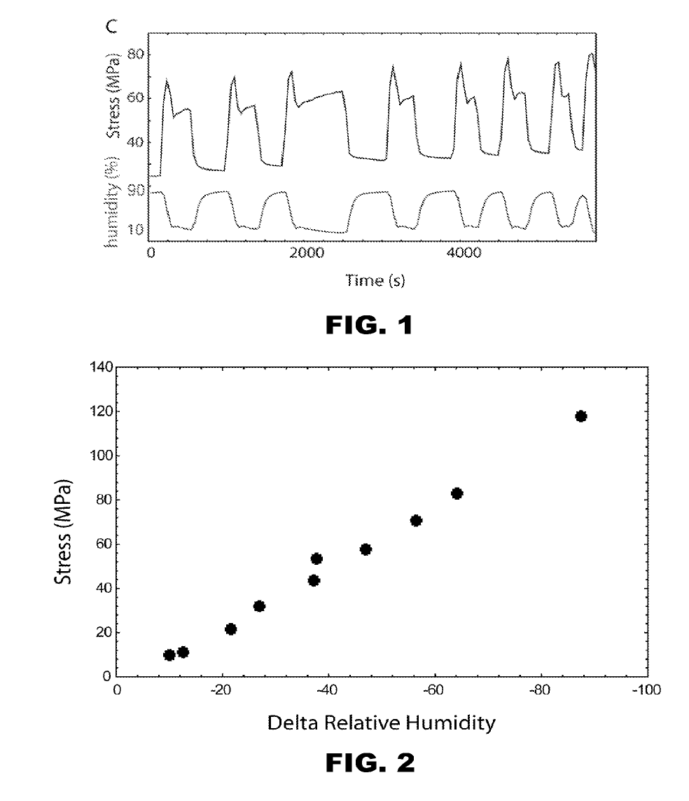

As an example of the characteristics of a fibrous material according to the invention, characteristics of certain dragline silk is shown in FIG. 1. In FIG. 1, a single 5 μm diameter dragline thread from the golden silk orbweaver Nephila clavipes exhibits a cyclical response to changes in humidity. For example, the charted behavior may occur after a fiber is first supercontracted by exposure prior to a threshold relative humidity (RH), for instance about 70% relative humidity. Such response may then be measured after mounting the dry silk at an initial strain of 0.5%, then cycling between about 10% RH and about 90% RH. Measurements may be made, for example, using a MTS Nano Bionix which measures force to ±10 μN, with the fiber being disposed in and custom fit with an environmental chamber which controls humidity to an accuracy of 1% with a range of 1-95% relative humidity. The test was conducted on an initial drying induced stress (contraction) of approximately 40 MPa, although the range of drying induced stress may be between 10 and 140 MPa. As shown in FIG. 1, the contracted fiber may then relax back to its post-supercontraction tension when humidity is subsequently increased.

As is generally known in the art, spider silk comprises one or more amino acids, namely alanine alone or alanine in combination with glycine, and in dragline silk, also proline. By varying the ratio of alanine, glycine, and proline when synthesizing silk fiber, it is possible to vary the tensile strength and density of the synthesized silk. It is also possible to control the orientation and amount of crystalline and amorphous biopolymer regions which comprise the synthesized spider silk by varying the silk synthesizing conditions, including pH, pressure, and speed of extrusion. As such, the humidity response and corresponding contraction and relaxation behavior of the synthetic silk may be controlled and customized to suit specific applications. Such processing steps may also be used to create synthetic silk having varying supercontracting humidity thresholds.

This cyclic response of the silk, whether natural or synthesized, may occur both prior to and after supercontraction, generating high forces in the silk fiber. While this cyclic response may occur prior to supercontraction, in the present invention, it is envisioned that it may be beneficial to supercontract a fiber prior to incorporation into a system or use in a method according to the invention, and thereafter controlling the contraction and relaxation behavior in a predetermined manner, without the possibility of supercontraction occurring. It is also envisioned that the characteristics of supercontraction could be employed in systems or methods to take advantage of the significant contraction and forces or displacement associated with this, though it may be a single-occurrence phenomenon. For example, it may be possible to control the cyclic behavior of the silk fiber material prior to supercontraction using a limited range of operative relative humidities below the supercontraction threshold relative humidity level, for example below about 70% relative humidity, and then if needed or desired to selectively cause supercontraction by elevating the RH to the supercontraction threshold. In such an instance, the fiber may be made to contract over relative humidity ranges between 0% and 65% to avoid supercontraction, thereby limiting the potential range of contraction and relaxation. Alternatively, the fiber material may initially be supercontracted and then the cyclic contraction/relaxation characteristics may be employed without concern in relation to supercontraction or the RH levels used for implementing the cyclic response characteristics relating thereto. Thus, a post-supercontracted fiber could be exposed to humidity ranges between 0% and 100% without the possibility of undesired or unexpected supercontraction occurring.

As shown in FIG. 1, the fiber may exhibit no evidence for fatigue in the silk after eight cycles, even after approximately 100 minutes under tension. As further shown in FIG. 2, the magnitude of the stress generated may be directly proportional to the change in humidity, providing a precise mechanism to control the stress generated by the spider silk.

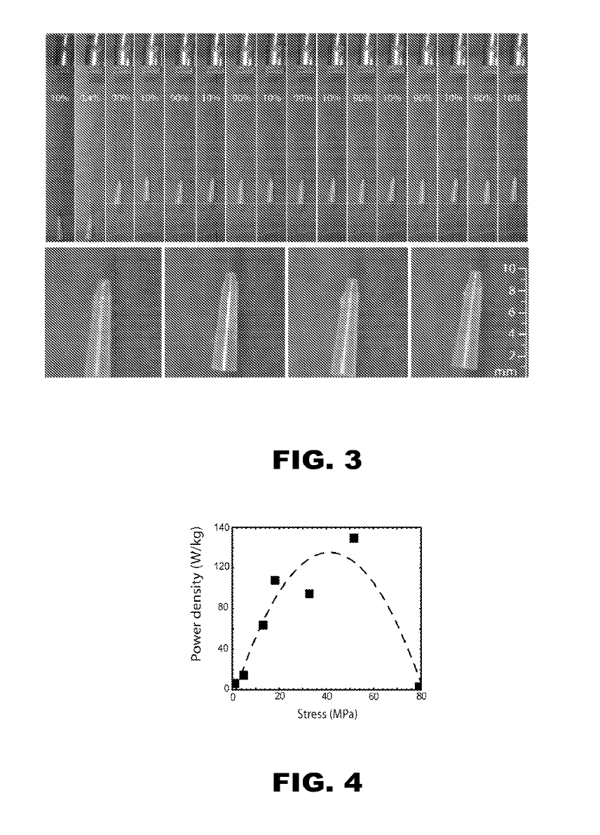

The cyclic contraction of silk fiber can produce work, which may be sufficient for a single 5 μm diameter fiber to lift at least 100 mg. As shown in FIG. 3, a single fiber may be used to lift a 9.5 mg weight in response to seven cycles of drying in the environmental chamber. During such cycling, the average displacement was 0.65 mm, or 1.7% of the fibers post-supercontraction length. The lifting response may occur within 3 seconds, and this response time may be reduced by increasing the rate of change in humidity or increased by decreasing the rate of change in humidity or tailoring the fiber characteristics during synthesis.

The power generated as a function of applied stress is shown in FIG. 4. A power density of about 150 W/kg may be achieved at a stress of about 50 MPa, which may be comparable to a typical power density for human muscles. In addition, the silk's sustainable stress of 80 MPa is much higher than 0.1 MPa for biological muscles. Furthermore, the energy density of silk fibers has been measured to be 0.54 J/cm³, which is fifty times greater than that of typical biological muscles.

The maximum sustainable stress, work density, power density, and modulus for silk are superior to synthetic polymer-based muscle mimics, as shown in the following table:

| | | | | Liquid | | |
|---|---|---|---|---|---|---|
| | | | Polyurethane | crystal | Conductive | Ferroelectric |
| Properties | Muscle Fiber | Silk Fiber | Electrostrictive | Polymers | Polymers | Polymers |
| Sustainable | 0.1⁴-0.35¹⁹ | 80 | 1.9 | 0.45 | 5-34 | 20 |
| Stress (MPa) | | | | | | |
| Work density | 0.008 (typical) | 0.54 | 0.1 | 0.003-0.056 | 0.1 | 0.3 |
| (J/cm³) | −0.04 (max) | | | | | |
| Power density | 50-300 | 150*-240* | — | 1 | 150 (max) | — |
| (W/kg) | | | | | | |
| Strain (%) | 20⁴, >40¹⁹ | 2.5 | 11 | 19-45 | 2-12 | 3.5 |
| Modulus | 10-60 | 18000 | 17 | 4 | 800 | 400 |
| (MPa) | | | | | | |
| Mechanism | chemical | humidity | voltage | Voltage | voltage | voltage |

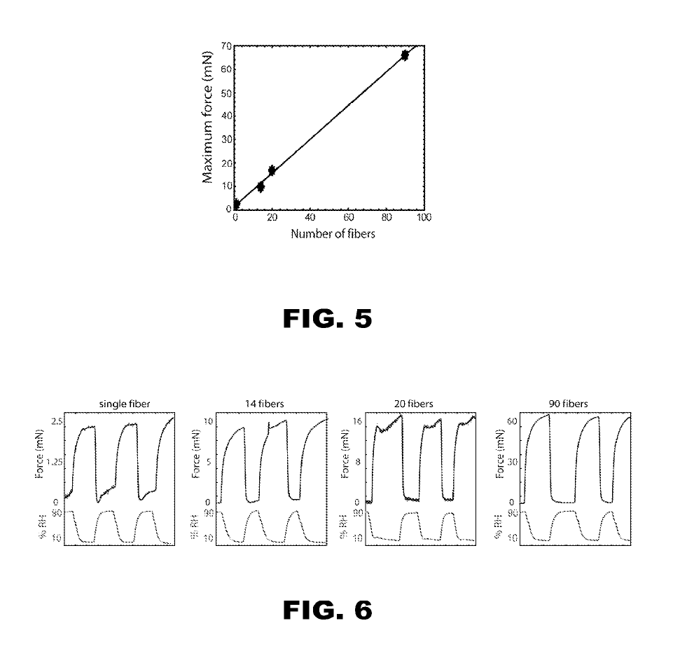

It is also contemplated that the silk fibrous material according to the invention can be formed in scaled up forms, such as bundles of fibers, sheets of fibers, coils of fibers or many other configurations using multiple fiber elements. Natural spider silk may similarly be used in a plurality of configurations. The force lifted by a single silk fiber can thus be easily scaled up by forming bundles, sheets or other forms of silk fibers, with results as shown in FIG. 5 relating to bundles of fibers. The orientation of fibers in a matrix may also be effectively used to perform various functions in a system or method in conjunction with other fibers in the matrix. Further, a system or method may also take advantage of exposing only a portion of a fiber, bundle, sheet or other body of fibers or the like, to RH changes, to thereby impart contraction/relaxation to such part without affecting other portions of the body of fibers. As shown in FIG. 6, bundles of 1-90 silk fibers lift forces that scale directly with the number of fibers. It is estimated that a silk bundle equivalent in cross-sectional area to a one mm diameter fiber may be able to lift as much as five kg while the equivalent of a two cm diameter fiber may be able to lift two metric tons, making silk fibrous bodies useful for a range of applications from MEMS devices to macro-robotics. However, the materials that exhibit higher strains may have substantially lower sustainable stress and modulus compared to spider silk. As there is considerable variation in silk produced by different species of spiders, bio-prospecting may reveal silk fibers that maintain higher cyclic strain without compromising other properties, which may be synthesized, or unique characteristics may be imparted using different synthesis and manufacturing techniques or processes. Furthermore, it is envisioned that synthetic silk exhibiting high strains as well as high sustainable stress as compared to natural spider or other organism silk may be produced.

The substantial power generated by silk fibers means that higher effective displacements can be achieved through strain amplification from engineering design. Although the displacement that may be achieved may be approximately 2.5% strain by varying the relative humidity to which the silk fiber is exposed, it may be possible to join multiple fibers or chains thereof in end-to-end fashion, whereby the 2.5% strain may be compounded across the multiple fibers to increase the overall strain. It is also envisioned that the silk may be woven into mats, such that it may contract in two dimensions. It is still further envisioned that the silk may be formed into three-dimensional articles, such that the article may contract in width, length, and height.

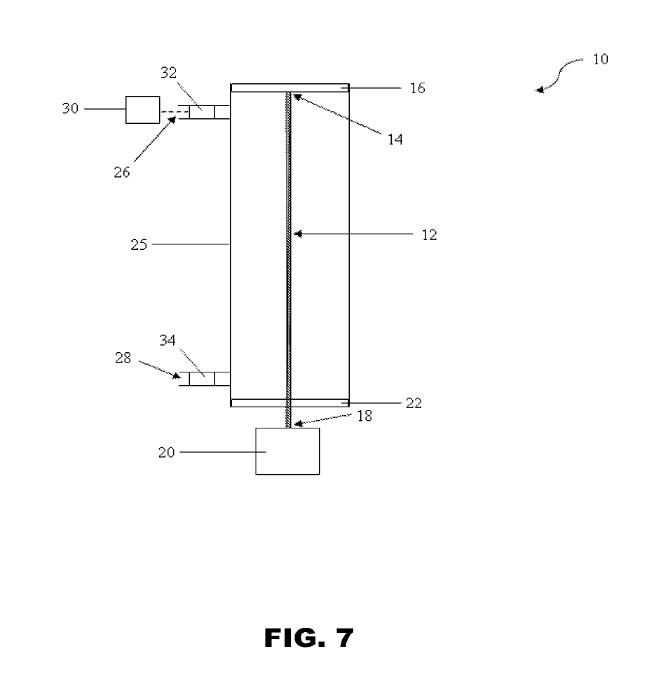

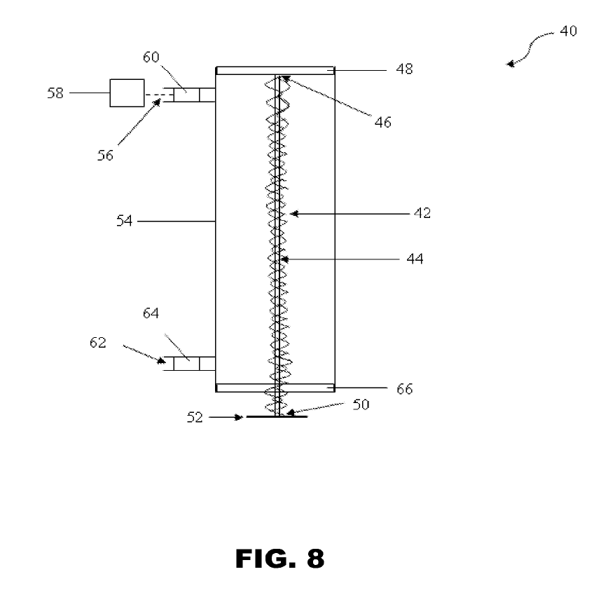

As merely examples of possible systems for performing work, FIGS. 7 and 8 show schematic illustrations of systems using one or more silk fibers, which may be natural or synthetic. In FIG. 7, the system 10 may include a bundle off silk fibers 12, with a first end 14 restrained in association with a support surface 16. The other end 18 of the bundle 12 may be attached to a displaceable member 20 through a barrier 22. The bundle 12 may be positioned within an atmosphere controlled chamber 25, having an inlet port 26 and an outlet port 28 as an example. The inlet port 26 may be selectively coupled to a relative humidity atmosphere source 30 for selectively introducing an atmosphere of a given relative humidity into the chamber 25. The source 30 may be any suitable system to provide a humidity-controlled environment. By humidity-controlled environment, it is meant an environment, the humidity of which is only governed by the humidity of the air or gas which is blown into the environment and is not subject to the ambient atmospheric relative humidity. Wet air having a first humidity and dry air having a second humidity may be alternately passed through the humidity controlled environment, wherein the first humidity is a higher relative humidity than the second humidity, or mixed and introduced in a controlled manner. A suitable inlet valve 32 may be used to selectively introduce the predetermined relative humidity atmosphere into the chamber 25, and valve 34 used to selectively evacuate the chamber 25 of a predetermined relative humidity atmosphere, either while introducing a different relative humidity atmosphere through inlet port 26 or separately as desired. In this manner, the relative humidity atmosphere in the chamber 25 can be precisely controlled and therefore the contraction/relaxation characteristics of the fibers in the bundle 12 can be controlled in a predetermined manner. Other configurations to introduce and alter the relative humidity atmosphere in the chamber 25, as well as other methods of exposing the silk fiber(s) 12 to predetermined RH to control the contraction/relaxation characteristics thereof are contemplated. In this example, the controlled contraction/relaxation characteristics of the fiber(s) 12 impart desired displacement to the displaceable member or element 20.

In this or similar embodiments, the system can perform predetermined work in a system, and may use a post-supercontraction silk fiber body having a first end and a second end, and a lifting fixture, such that a first end of the silk fiber body is connected to the first support surface and the second end of the silk fiber body connected to the an element to be displaced. In operation, exposing the silk fiber body to a humidity-controlled atmosphere causes the silk fibers to contract and relax in a predetermined manner, such that the displaceable member or element is moved between at least first and second positions from such cyclic motion of the fiber body. Upon relaxation of the fiber body, the displacement may be caused by gravity or another force imposed upon the displaceable body, such as a spring force or the like. Such displacement may be useful in a wide variety of applications and devices, such as merely examples, sensors, actuators, micromachines, MEMS devices, motors, valves, switches, robotics, smart structures, toys, biomimetic muscles, prosthetics, catheters or other medical devices, surgical instruments, smart textiles or a myriad of other devices, systems, methods or applications.

In FIG. 8, an alternative system is shown schematically, taking advantage of the compounding strain that may be achieved in use of the silk fibers, wherein a system 40 comprises a silk fiber body 42 coiled about a support 44, to increase the effective length of the fiber body 42. It is envisioned that this silk may be either naturally occurring or synthesized. The first end 46 of fiber body 42 may be restrained on a support surface 48, with the second end retrained on an actuation surface 52. The fiber body may be positioned in a atmosphere controlled chamber 54 or other suitable arrangement to selectively expose the fiber body to a predetermined relative humidity atmosphere as described. The chamber may have an inlet port 56 selectively coupled to a relative humidity atmosphere source 58, with a valve 60 controlling injection of the predetermined relative humidity atmosphere into the chamber 54. An outlet port 62 and control valve 64 may be used to control evacuation of a relative humidity atmosphere from chamber 54. In this manner, the relative humidity atmosphere in the chamber 54 can be precisely controlled and therefore the contraction/relaxation characteristics of the fibers in the body 42 can be controlled in a predetermined manner. Other configurations to introduce and alter the relative humidity atmosphere in the chamber 54, as well as other methods of exposing the silk fiber(s) 42 to predetermined RH to control the contraction/relaxation characteristics thereof are contemplated. In this example, the controlled contraction/relaxation characteristics of the fiber(s) 42 impart desired strain or force on the actuation surface 52. Such force or strain may be useful in a wide variety of applications and devices, such as merely examples, power generation, sensors, actuators, micromachines, MEMS devices, robotics, smart structures, toys, biomimetic structures, prosthetics, medical devices or a myriad of other devices, systems, methods or applications.

In one embodiment, it is envisioned that a not-yet-supercontracted silk fiber or body of fibers, whether natural or synthetic, may be fixedly attached in tension between two surfaces forming a sensor-type device. At least one of the two surfaces may comprise a piezoelectric material, such that applying a force to the piezoelectric material will result in production of a corresponding electric signal. In operation, the device may be placed in a humidity controlled environment, wherein the relative humidity level must not rise above a certain level. For example, some electronic equipment may only operate in an environment where the relative humidity cannot rise above a predetermined level. A synthetic silk material may be produced having a threshold supercontraction level which corresponds the predetermined relative humidity level or corresponds to a second predetermined level that is lower than the predetermined humidity level. Alternatively, a natural spider silk having a known supercontraction threshold may be used. It is envisioned that the sensor device may be coupled to an alarm system, such that when the silk is exposed to the threshold humidity, it supercontracts, thereby transferring a force to the piezoelectric substrate. In turn, the piezoelectric substrate generates an electrical signal, which is coupled to an alarm. As the alarm detects the electrical signal, it alerts an individual that the relative humidity of the controlled-humidity environment has reached the predetermined humidity level. It is envisioned that a post-supercontraction silk may also be used to measure changes in relative humidity in a similar fashion. Such a system could also generate power by imposition of strain on piezoelectric material to which the silk fiber body is attached. Other applications and uses for the imposed strains produced by cyclic contraction/relaxation of the silk fiber body are contemplated. Changes in relative humidity can be controlled or the changes in the local environment that occur normally can be used to activate the silk fiber contraction/relaxation characteristics, allowing passive or active control thereof.

In a further embodiment, post-supercontraction spider silk, either natural or synthetic, may be woven into a bandage, which may be applied to a wound. As the wound bleeds, the silk may contract in response to the increase in humidity, thereby creating a tourniquet effect upon the wound and decreasing the blood flow therefrom. As the bleeding rate subsides or ceases, the bandage may dry out and correspondingly, retract. Should the bleeding begin to increase again, the bandage may again contract in response to the humidity increase.

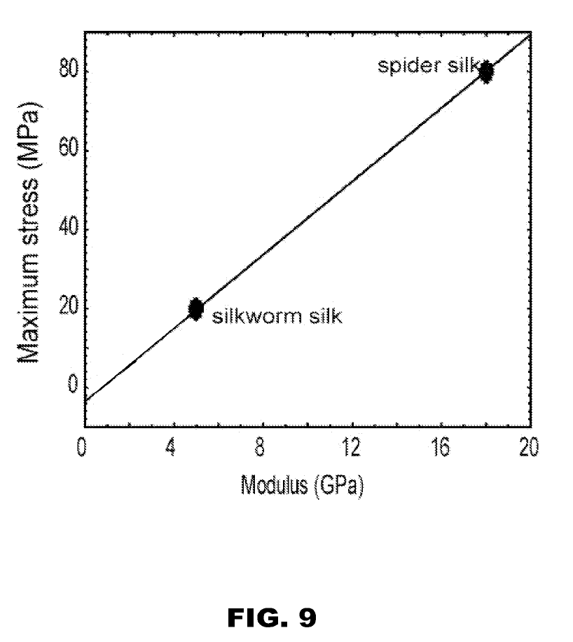

Turning to FIG. 9, there is shown a graph relating to the cyclical responses of contraction and relaxation of silk materials associated with spider silk as well as silk produced by silkworms. The maximum stress vs. the modulus is shown for silkworm silk and spider silk, each showing a cyclic response to humidity. Though the characteristics of the spider silk indicate increased response to humidity, the response is also seen to be characteristic of silkworm silk, and may be used according to the invention.

The characteristics of the silk material may also be modified by suitable treatment thereof, such as by making it stronger by reinforcement using coatings, such as coating formed of metal oxide materials, such as aluminum oxide (Al₂O₃), titanium oxide (TiO₂) or zinc oxide (ZnO). Other treatment processes may allow for manipulation of the cyclical response characteristics of the material. The addition of metal based particles to the silk threads may allow for modification of the response characteristics of the individual threads or a bundle of threads for example, such as making the contraction response greater, controlling response rates (contraction and/or relaxation), triggering responses (contraction and/or relaxation) at predetermined humidity levels or the like. Treatment of the individual threads or bundle of threads by suitable techniques to control the response characteristics thereof are thus contemplated within the scope of the invention.

The invention has been described herein with reference to the disclosed embodiments. Obviously, modifications and alterations will occur to others upon a reading and understanding of this specification. It is intended to include all such modifications and alterations insofar as they come within the scope of the appended claims or the equivalence thereof.

1. The fibrous body of claim 7, wherein the at least one silk fiber is formed of an aqueous solution of a silk protein formed to have predetermined contraction/relaxation characteristics, wherein the contraction/relaxation characteristics are initiated by exposure thereof to predetermined relative humidity characteristics in the adjacent atmosphere.

2. The fibrous body of claim 7, wherein the at least one silk fiber has contraction characteristics including supercontraction, where the fiber contracts a percentage of its length upon being exposed to a predetermined relative humidity.

3. The fibrous body of claim 1, wherein the contraction/relaxation characteristics are cyclic.

4. (canceled)

5. The fibrous body of claim 1, wherein the composition is synthesized in an extrusion process, and the orientation and amount of crystalline and amorphous biopolymer regions which comprise the synthesized material are controlled using synthesizing conditions selected from the group consisting of pH, pressure, and speed of extrusion or combinations thereof.

6. (canceled)

7. A fibrous body formed of at least one silk fiber, the at least one silk fiber having predetermined contraction/relaxation characteristics, wherein the contraction/relaxation characteristics are initiated by exposure thereof to predetermined relative humidity characteristics in the adjacent atmosphere, wherein the fibrous body will have predetermined contraction/relaxation characteristics resulting from the exposure thereof to predetermined relative humidity characteristics in the adjacent atmosphere.

8. The fibrous body of claim 7, wherein the body is selected from the group consisting of a bundle of fibers of a predetermined size or diameter, a meshwork of fibers forming a predetermined configuration such as one or more sheets or bundles, coils of fibers or combinations thereof.

9. A system comprising at least two members between which at least one fiber is attached to impart a predetermined force or displacement between the at least two members, the at least one fiber having predetermined contraction/relaxation characteristics, and a controlled atmosphere at least in the vicinity adjacent to at least a portion of the at least one fiber, such that the predetermined contraction/relaxation characteristics of the at least portion of the at least one fiber are controllable by control of the relative humidity in the atmosphere adjacent the at least one fiber.

10. The system of claim 9, wherein the contraction/relaxation characteristics of the at least one fiber are cyclic.

11. (canceled)

12. The system of claim 9, further comprising a chamber in which the at least one fiber is housed and in which the relative humidity is controllable.

13. (canceled)

14. The system of claim 12, wherein the chamber has an inlet port, with the inlet port is selectively coupled to a relative humidity atmosphere source for selectively introducing an atmosphere of a given relative humidity into the chamber.

15. The system of claim 14, wherein the relative humidity atmosphere source comprises a source of wet air having a first humidity and a source of dry air having a second humidity, with the sources of wet and dry air selectively introduced into the chamber separately or after mixing.

16. The system of claim 14, wherein the inlet port has a valve associated therewith to control ingress or egress of gases therethrough.

17. The system of claim 9, wherein the at least one fiber is attached to a stationary member and at least one movable member.

18. The system of claim 17, further comprising a chamber in which the at least one fiber is housed and in which the relative humidity is controllable, and wherein at least movable member is positioned outside of the chamber.

19. (canceled)

20. (canceled)

21. (canceled)

22. A method of using at least one silk fibrous body by attaching the at least one fibrous body to at least two members to selectively impart a force on at least one member or displacement of at least one member by subjecting the at least one fibrous body to an atmosphere having predetermined relative humidity characteristics, wherein the predetermined relative humidity characteristics cause predetermined contraction or relaxation of the at least one fibrous body.

23. The method of claim 22, wherein the at least one fibrous body has contraction/relaxation characteristics are cyclic.

24. (canceled)

25. (canceled)

26. (canceled)

27. (canceled)

28. The method of claim 22, wherein the at least one silk fibrous body is synthesized in an extrusion process and the contraction/relaxation characteristics thereof are controlled by controlling the process in a manner selected from the group consisting of controlling the orientation of crystalline and amorphous biopolymer regions in the body, controlling the amount of crystalline and amorphous biopolymer regions in the body or combinations thereof.

29. The method of claim 28, wherein the step of controlling the orientation and/or amount of crystalline and amorphous biopolymer regions in the body are performed by varying the synthesizing conditions selected from the group consisting of pH, pressure, speed of extrusion or combinations thereof.

30. The method of claim 22 further comprising positioning the at least one fibrous body in a chamber in which the at least one fiber is housed and in which the relative humidity is controllable.

31. (canceled)

32. (canceled)

33. (canceled)

34. (canceled)

35. The method of claim 22, wherein the at least one fibrous body is attached to a stationary member and at least one movable member.

36. (canceled)

37. (canceled)

38. (canceled)
